(19) United States
(12) Patent Application Publication
(10) Pub. No.: US 2024/0377386 A1
(43) Pub. Date: Nov. 14, 2024

(54) DIAGNOSTIC MICROFLUIDIC CARTRIDGE

(71) Applicant: CFD Research Corporation, Huntsville, AL (US)

(72) Inventors: David Edwin Gaddes, III, Huntsville, AL (US); Trevor Jaxon Suddarth, Huntsville, AL (US); Ketan Harendrakumar Bhatt, Huntsville, AL (US)

(21) Appl. No.: 18/659,540

(22) Filed: May 9, 2024

Related U.S. Application Data
(60) Provisional application No. 63/501,322, filed on May 10, 2023.

(51) Int. Cl. G01N 33/52; B01L 3/00

(52) U.S. Cl. CPC G01N 33/52; B01L 3/502715; B01L 2200/027; B01L 2200/16; B01L 2300/044; B01L 2300/0672; B01L 2300/1805

(57) ABSTRACT

A diagnostic system is provided, which can include a sample tube having an input end and an output end, a microfluidic device, a heating member, and a reagent. The microfluidic device may include a port member configured to couple with the sample tube, a sample distribution member coupled with the port member and having a microfluidic network with microfluidic channels, reaction chambers fluidly coupled with the microfluidic channels, a top cover configured to receive the sample tube therein and having openings corresponding to the reaction chambers, and a bottom member configured to mate with the top cover to enclose the port member and the sample distribution member. The bottom member may include a bottom chamber for retaining a composition. The heating member may be placed in the bottom chamber, and the reagent in the reaction chambers may provide a colorimetric reading upon interaction with a target analyte in the sample.

### CROSS-REFERENCE TO RELATED APPLICATIONS

This patent application claims priority to U.S. Provisional Application No. 63/501,322 filed May 10, 2023, which is incorporated herein by specific reference in its entirety.

### U.S. GOVERNMENT RIGHTS

This invention was made with government support under W81XWH-22-C-0026 awarded by the Department of Defense. The government has certain rights in the invention.

### BACKGROUND

### Field

This invention relates to a diagnostic system that is configured to test a sample with respect to reagents. More specifically, the present invention pertains to diagnostic systems that are configured to test a sample in an analysis protocol that requires heat. The diagnostic system according to one or more embodiments of the present disclosure may perform the heated analysis protocol without the use of any mechanical or electrical heating device therewith.

### Description of Related Art

Biological samples may be analyzed using reagents to detect, identify, or quantify presence of particular substances in the sample. The reagents may be selected based on the ability to react selectively with certain target substance(s) in the samples. The samples may be mixed with the selected reagents, and the results may be observed to identify the presence of the target substances. For example, in some instances, the presence of target substances in the sample may be represented as various colors. In some instances, the results may be represented using different reactions, such as changes in characteristics of the samples. The identification of the target substances may allow detection of different diseases and/or conditions that may be present in the samples and corresponding sources of the samples.

Heating mechanisms may be commonly used for enhancing and/or initiating the reactions between the samples and the reagents. For example, heating the mixture of the samples and the reagents may accelerate the reactions, increase sensitivity of the reactions, enhance solubility of the mixtures, eliminate interfering substances from the sample through evaporation, and/or control the reaction rates, among others.

However, the heating mechanisms often add a layer of burden to the analysis process of the samples. Sample analysis models incorporating heating mechanisms require certain settings that may lead to use of the heating mechanism burdensome. For example, common heating mechanisms may include heated water, block heater, heating mantle, hot plate, incubator, microwave digestion systems, flame burner, among others. Use of such heating mechanisms may be difficult as the heating mechanisms are not portable, require external power sources (e.g., mechanical and/or electrical), may not be safe (e.g., use of flames), and/or may not be efficient.

In summary, a diagnostic system that provides efficient and portable heating to the samples without use of mechanical or electrical heating device does not exist. However, such a diagnostic system is desirable.

### SUMMARY

In some embodiments, a system for analyzing a sample may include a sample tube having a removable lid and an output end and a microfluidic device. The microfluidic device may include a port member configured to couple with the output end of the sample tube; a sample distribution member coupled with the port member and having a microfluidic network with one or more microfluidic channels; one or more reaction chambers fluidly coupled with the one or more microfluidic channels, in which the reaction chambers include readout regions corresponding to each reaction chamber; a top cover with a main slot configured to receive the sample tube therein, and with one or more openings corresponding to the one or more reaction chambers; and a bottom member mating with the top cover to enclose the port member and the sample distribution member, in which the bottom member includes a bottom chamber configured to retain a composition. The system may further include a heating member configured to be placed into the bottom chamber and a reagent in the one or more reaction chambers that is adapted to provide a colorimetric reading upon interaction with a target analyte in the sample.

However, the diagnostic system performs the heated analysis protocol without the use of any mechanical or electrical heating device therewith. The current diagnostic system is configured for receiving a biological sample and testing the biological sample in one or more analytical reaction chambers under heating supplied by a self-heating phase change material, such as a salt hydrate composition, or an electrical heater powered by a battery. However, environmental samples (e.g., water) or other types of samples (e.g., industrial liquids) can be used in the device with the self-heating phase change material or electric heater providing the heat for the analytical protocol. Accordingly, the heat required for performing the reaction of the analytical protocol can be provided by the phase change material, which can include a eutectic salt hydrate. However, a battery or plugged power supplies for electric heaters can also be used.

In some embodiments, the port member has a conical body with a tapered input tip. In these and other embodiments, the output end of the sample tube include a port adapter configured to couple with a port of the microfluidic device. In some embodiments, the output end of the sample tube may include a film covering the port adapter that includes a puncturable membrane material that can be punctured with the tapered input tip of the port member, such as an elastomer, rubber, or thermoplastic.

In some embodiments, the port may include the port member and an annular receiver, in which the annular receiver is configured as an alignment port having an annular recess configured for receiving an annular recess configured for receiving an annular lip of the output end of the sample tube.

In some embodiments, the sample tube may be configured for receiving a swab, and the reaction tube may be configured for passing the content of the swab to the port member. In some embodiments, the sample tube may include a spout at the input end, in which the removable lid is configured to cover the spout.

In some embodiments, the microfluidic device may include an even number of the reaction chambers. In other embodiments, the microfluidic device may include an odd number of the reaction chambers. In some embodiments, the reaction chambers may protrude from the sample distribution member through one or more openings such that the readout regions are viewable through the one or more openings. In some embodiments, the system may further include a plunger adapted to fit into the sample tube and function as a syringe for delivering the sample through the microfluidic network into the reaction chambers.

In some embodiments, the reagent may be configured to test for a sexually transmitted disease with a colorimetric assay. Additionally or alternatively, the reagent may be configured to test for other types of diseases and/or conditions that may be tested using the reagent.

In some embodiments, the heating member may include a eutectic salt composition including a salt hydrate having a phase transition temperature between about 37° C. and about 90° C., between about 38° C. and about 80° C., between about 39° C. and about 75° C., between about 40° C. and about 70° C., between about 50° C. and about 65° C., or about 60° C. to about 65° C. In some embodiments, the salt hydrate may be 50% Mg(NO₃)₂ 6H₂O+50% Al(NO₃)₂ 9H₂O with a phase transition temperature of about 65° C. In some embodiments, the heating member may include an electronic heater, whether battery powered or plugged into a power outlet.

In some embodiments, a method of analyzing a sample may include: obtaining a sample in a sample tube, the sample tube having an input end with an input opening where the sample is obtained through, the sample tube also having an output end having a port adapter, the port adapter configured to release the sample in response to being punctured; attaching the port adapter of the sample tube to a port member of a microfluidic device, the port member having a conical body with a tapered input tip adapted to couple with the port adapter, and having an output end with a port conduit extending between a tip opening at the input tip to an output opening at the output end, wherein the tapered input tip is configured to puncture the port adapter to obtain the sample from the sample tube; distributing the sample to one or more reaction chambers, by a sample distribution member having a microfluidic network with one or more microfluidic channels, the one or more microfluidic channels having a microfluidic channel inlet fluidly coupled with the output opening at the output end of the port member, and having a microfluidic channel outlet fluidly coupled with the one or more reaction chambers; providing heat to the one or more reaction chambers by a heating member in a bottom chamber under the one or more reaction chambers; interacting the distributed sample with a reagent pre-deposited in each reaction chamber, the reagent adapted to provide a colorimetric reading upon interaction with a target analyte in the sample; and reading the colorimetric reading of each reaction chamber through a readout region of each reaction chamber.

In some embodiments, the sample may be obtained using a swab, in which the swab is used to obtain the sample from a subject. In some embodiments, the heat may be provided to the one or more reaction chambers using a heating member.

In some embodiments, a method of manufacturing a sample analyzing system may include: obtaining a bottom member, the bottom member including a bottom chamber, the bottom chamber surrounded by one or more support rods configured to support a bottom platform; placing a heating member into the bottom chamber; placing a sample distribution member on the bottom platform, the sample distribution member coupled with a fluidic seal to fluidly seal one or more microfluidic channels of a microfluidic network within the sample distribution member, the sample distribution member also having a port member fluidly coupled with the one or more microfluidic channels, the port member having a conical body with a tapered input tip; attaching one or more reaction chambers to the sample distribution member, the one or more reaction chambers fluidly coupled with the one or more microfluidic channels, each reaction chamber containing a reagent that is adapted to provide a colorimetric reading upon interaction with a target analyte, the one or more reaction chambers also placed on top of the bottom platform; covering the sample distribution member with a top cover, the top cover configured to mate with the bottom member to enclose the port member, the sample distribution member, and the fluidic seal, the top cover having a first upper portion and a second portion, wherein the first portion includes a main slot configured to receive a sample tube therein, and the second portion includes one or more openings corresponding to the one or more reaction chambers; and placing the sample tube through the main slot of the top cover to be removably coupled with the port member, the sample tube having an input end and an output end, the sample tube configured to obtain a sample to be analyzed through the input end, wherein the port member may connect with the output end of the sample tube, the port member configured to distribute the sample to the one or more reaction chambers through the sample distribution member.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes a bird-eye view of a diagnostic system including a sample tube and a microfluid reactor device;

FIG. 1B includes a top view of the diagnostic system of FIG. 1A;

FIG. 1C includes a side view of the diagnostic system of FIG. 1A;

FIG. 1D includes a bottom view of the diagnostic system of FIG. 1A;

FIG. 1E includes a front view of the diagnostic system of FIG. 1A;

FIGS. 2A-2B include different cross-sectional views of the diagnostic system of FIG. 1A;

FIG. 3A includes a bird-eye view of a sample tube attached to a microfluidic device including an annular receiver;

FIG. 3B includes a bird-eye view of the sample tube attached to the annular receiver

FIG. 3A;

FIG. 3C includes a bird-eye view of s ample tube attached to a microfluidic device without the annular receiver of FIG. 3A.

FIG. 4A includes a bird-eye view of a sample tube;

FIG. 4B includes a side view of the sample tube of FIG. 4A;

FIG. 4C includes a top view of the sample tube of FIG. 4A;

FIG. 4D includes a worm's eye view of the sample tube of FIG. 4A;

FIG. 4E includes a bottom view of the sample tube of FIG. 4A;

FIG. 4F includes a cross-sectional view of the sample tube of FIG. 4A;

FIG. 4G includes a swab that may be used to provide sample to the sample tube of FIG. 4A;

FIG. 5A includes a bird-eye view of a microfluidic device including a sample distribution member, a set of microfluidic channels and reaction chambers;

FIG. 5B includes a top view of the microfluidic device of FIG. 5A;

FIG. 5C includes a side view of the microfluidic device of FIG. 5A;

FIG. 5D includes a top view of the sample distribution member of FIG. 5A;

FIG. 5E includes a fluidic seal that is configured to be coupled with the sample distribution member of FIG. 5A;

FIG. 5F includes another embodiment of the sample distribution member;

FIG. 6A includes a bird-eye view of a top cover of the diagnostic system of FIG. 1A;

FIG. 6B includes a top view of the top cover of FIG. 6A;

FIG. 6C includes a front view of the top cover of FIG. 6A;

FIG. 6D includes a bottom view of the top cover of FIG. 6A;

FIG. 7A includes a bird-eye view of a bottom cover of the diagnostic system of FIG. 1A;

FIG. 7B includes a top view of the bottom cover of FIG. 6A;

FIG. 7C includes a front view of the bottom cover of FIG. 6A;

FIG. 7D includes a bottom view of the bottom cover of FIG. 6A;

FIG. 7E includes a bird-eye view of the bottom cover of FIG. 7A coupled with a platform member;

FIG. 8A includes a heating pouch having the phase change material;

FIG. 8B includes a heating pouch that can be punctured with a material that induces a phase change in a phase change material;

FIG. 9A includes a bird-eye view of an output cap, which may be an annular sample tube receiver when mounted in the microfluidic device;

FIG. 9B includes a top view of the output cap of FIG. 9A;

FIG. 9C includes a bottom view of the output cap of FIG. 9A; and

FIG. 10 includes an embodiment of the diagnostic system including an electric heater.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology relates to a diagnostic system that is configured to test a sample in an analysis protocol that requires heat. However, the diagnostic system performs the heated analysis protocol with the use of any heating member, whether mechanical or electrical heating device, or phase change material. The current diagnostic system is configured for receiving a biological sample and testing the biological sample in one or more analytical reaction chambers under heating. However, environmental samples (e.g., water) or other types of samples (e.g., industrial liquids) can be used in the device providing the heat for the analytical protocol.

The diagnostic system may include a sample tube configured to obtain the sample therein and a microfluidic device configured to perform the diagnostic analysis of the sample. The sample may include an input end and an output end, in which the input end may be closed with a removable lid. The sample tube may receive the sample through the input end. The output end of the sample tube may couple with the microfluidic device such that the sample receive through the input tube may be transferred to the microfluidic device through the output end. The output end of the sample tube may be sealed using a film. The film may be puncturable such that the microfluidic device may puncture the film to couple with the sample tube.

The microfluidic device may include one or more reaction chambers in which one or more reagents may be placed therein. The sample received through the sample tube may be distributed to the one or more reaction chambers to initiate reaction between the sample and the reagents. The sample may be distributed to the reaction chambers using a sample distribution member. The sample distribution member may include a network of microfluidic channels that are connected to the reaction chambers. The microfluidic channels may correspond to different reaction chambers such that the sample distributed to each reaction chamber is separated among the reaction chambers.

The sample distribution member and the one or more reaction chambers may be covered using a top cover and a bottom member. The top cover and the bottom cover may be configured to mate such that the top cover and the bottom member house the sample distribution member. The top cover may include a main opening configured to receive the sample tube, and one or more opening corresponding to the one or more reaction chambers. The one or more openings may allow observation of the reaction chambers in instances in which the microfluidic device is housed by the top cover and the bottom member.

The bottom cover may include a bottom member configured to retain a composition. The composition may include a heating member configured to provide heat to the one or more reaction chambers such that the temperature of the reactions within the reaction chambers may be controlled.

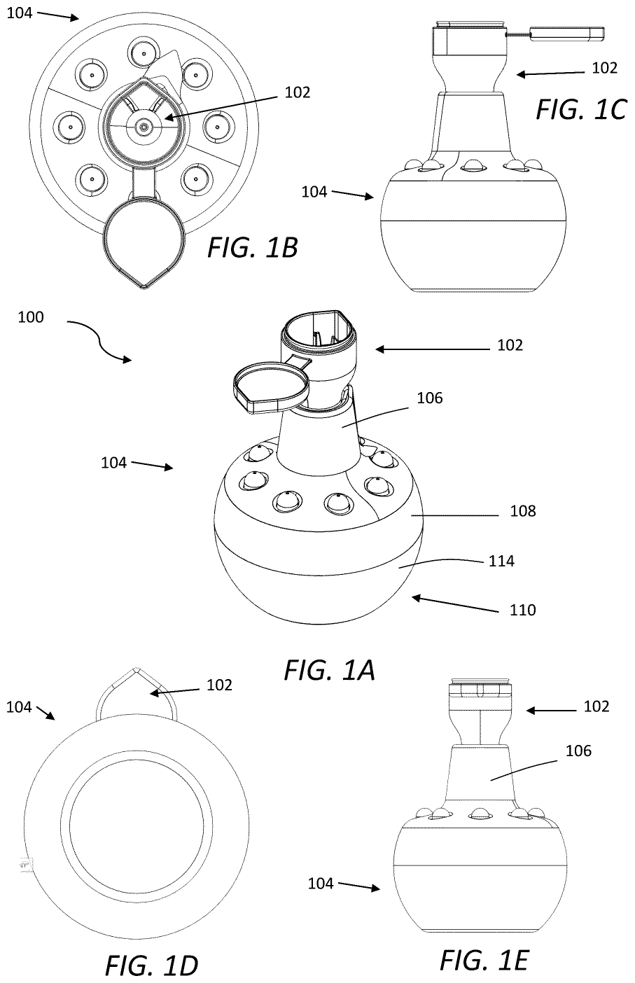

FIGS. 1A-1E illustrate an example embodiment of a diagnostic system 100 including a sample tube 102 and microfluidic reactor device 104. The microfluidic reactor device 104 can include a neck 108 adapted for receiving the sample tube 102. The neck 108 may be coupled to a body 110 having a top cover 112 and a bottom member 114.

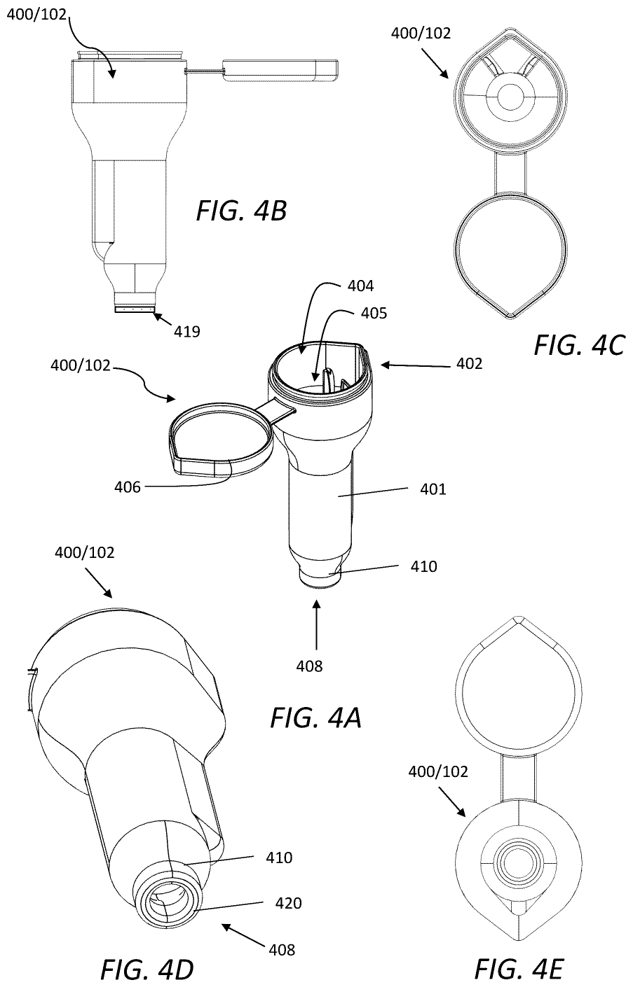

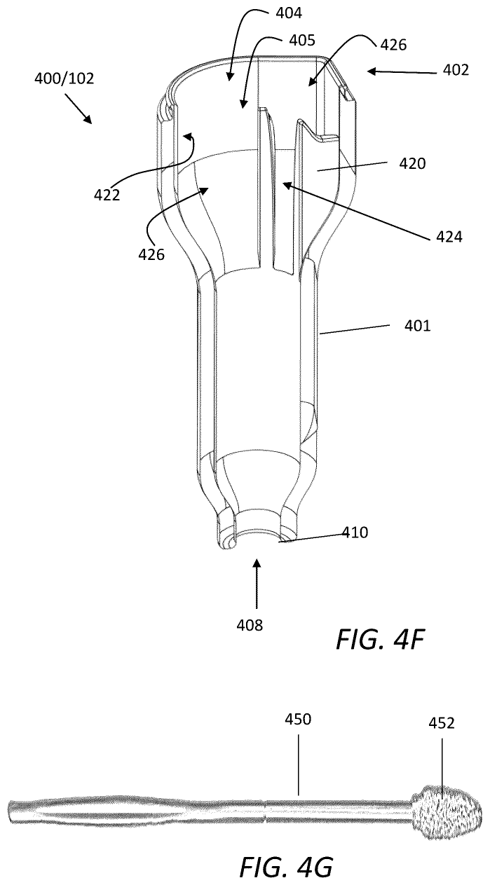

The sample tube 102 is illustrated in FIGS. 4A-4E as sample tube 400. The sample tube 400 is shown to include an input end 402 having an input opening 404 into a tube chamber 405 that is configured to receive the sample. In some embodiments, the input end 402 may include an attached lid 406 that is adapted to cover the input opening 404 to seal the input end 402 of the sample tube 400. In other embodiments, the lid 406 may not be physically attached to the input end 402. In some embodiments, the lid 406 may be removably attached to the input end 402. In some embodiments, the sample tube 400 may include an output end 408 opposite of the input end 402. In some embodiments, the output end 408 can be narrower than the input end 402. In some embodiments, the output end 408 may have a port adapter 410. The port adapter 410 may be adapted to release the sample received by the sample tube 102 through the port adapter in response to being punctured. The sample tube 400 includes a tube body 401 extending between the input end 402 and the output end 408, the tube body 401 being narrower than the input end 402 and being wider than the output end 408.

Also, FIG. 4B shows the sample tube 400 having a film 419 that covers the output end 408. In some embodiments, the film 419 may be composed of material that may be punctured, such as with the port member 502 having a conical body with a tapered input tip 504. That is, the tip 504 may puncture the film 419 so that the biological sample within the sample tube 400 can then pass from the sample tube 400 into the reaction device microfluidic pathway. In some embodiments, the end cap 900 is part of the sample tube 104, such as to cover the film 419.

FIG. 4F shows a cross-sectional view of the sample tube 400 with an internal tube chamber 405 that has pointed wings 420 protruding from the side walls 422 to form a narrow channel 424 between a smaller sub-chamber 426 and the main chamber 428. This structure allows for a swab, such as a swab 450 of FIG. 4G, to provide the biological sample by placing the swab 450 into the tube chamber 405 to use the wings 420 to squeegee, wring, constrict, or otherwise coax the biological sample from the swab head 452.

For example, the swab head 452 can be inserted into the tube chamber 405 and allow the biological sample to drip or otherwise transfer into the tube chamber 405. Additionally, the swab head 452 can be pressed against one or both wings 420 to coax the biological sample from the swab head. The narrow channel 420 can be used to pull or push the swab head 452 therethrough, such that the narrowness of the narrow channel 420 uses sides of the wings 420 to force out the biological sample from the swab head. Accordingly, the wings 420 can be used such that the biological sample can be from a swab, which can be easily used for mucosal, oral, buccal, vaginal, rectal, nasal, car, skin, or other swabbing to obtain a biological sample. Preferably, the biological sample includes nucleic acid material. FIG. 4G shows such a swab 450 having the swab head 452 with the biological sample. In some aspects, the narrow channel 420 can be tapered, such as being wider at a top and narrower at a bottom.

In some embodiments, an inert fluid can be used to push the biological sample through the microfluidic channels to the reaction chamber. That is, after the biological fluid is in the bottom of the sample tube, within the microfluidic pathway, or any place leading to the reaction chamber, an oil or silicone or other inert material can be pressed or pumped into the sample tube and then microfluidic channels in order to push the maximum amount of biological sample into the reaction chambers.

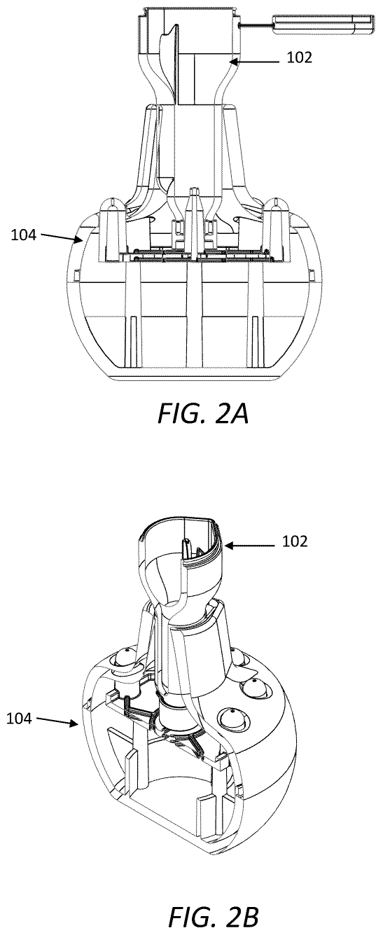

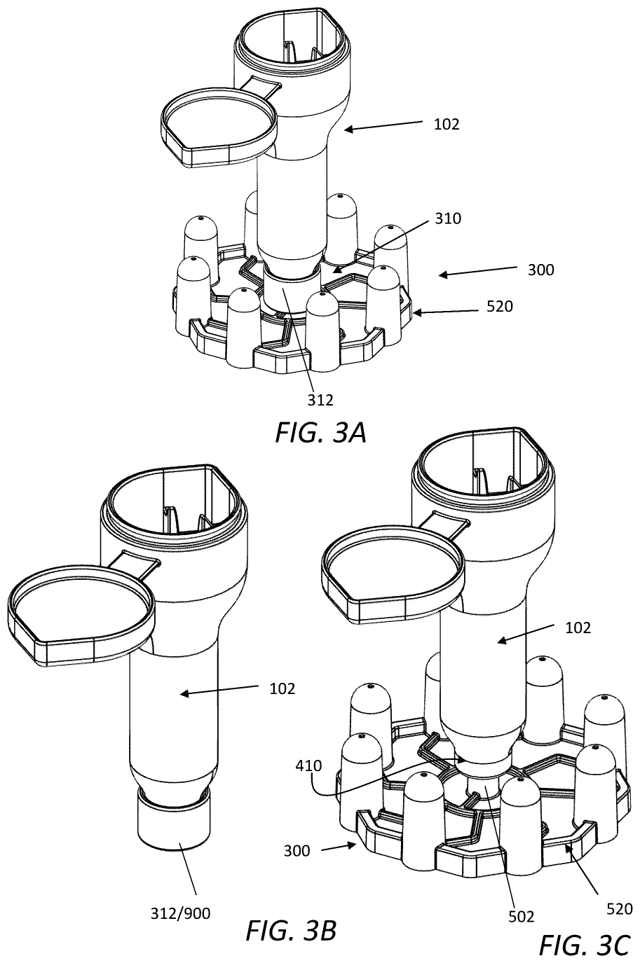

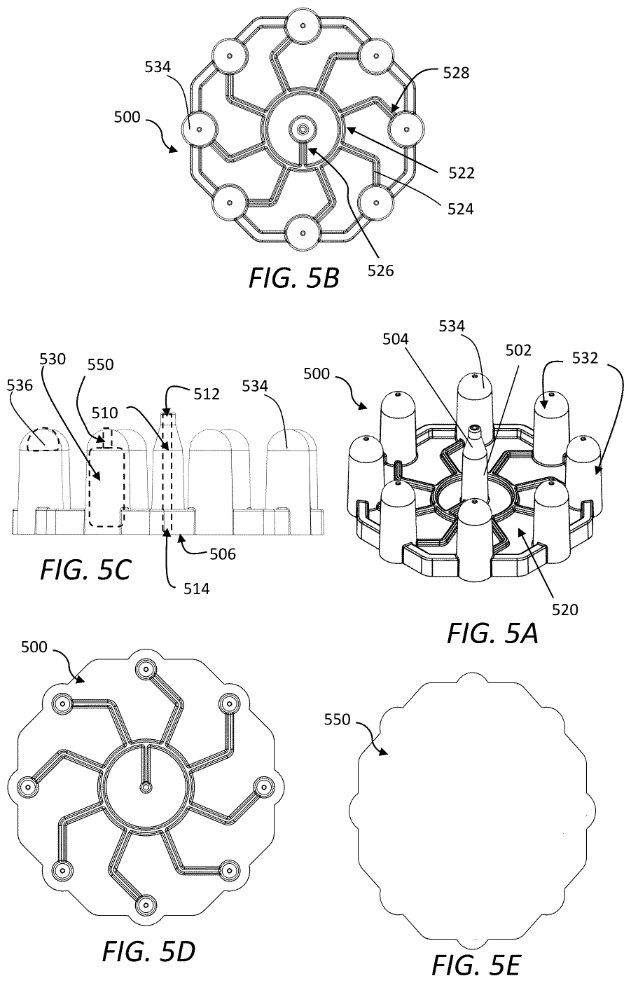

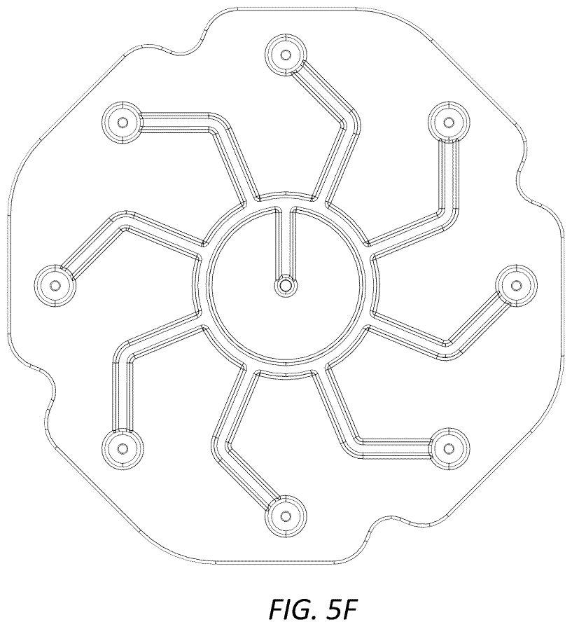

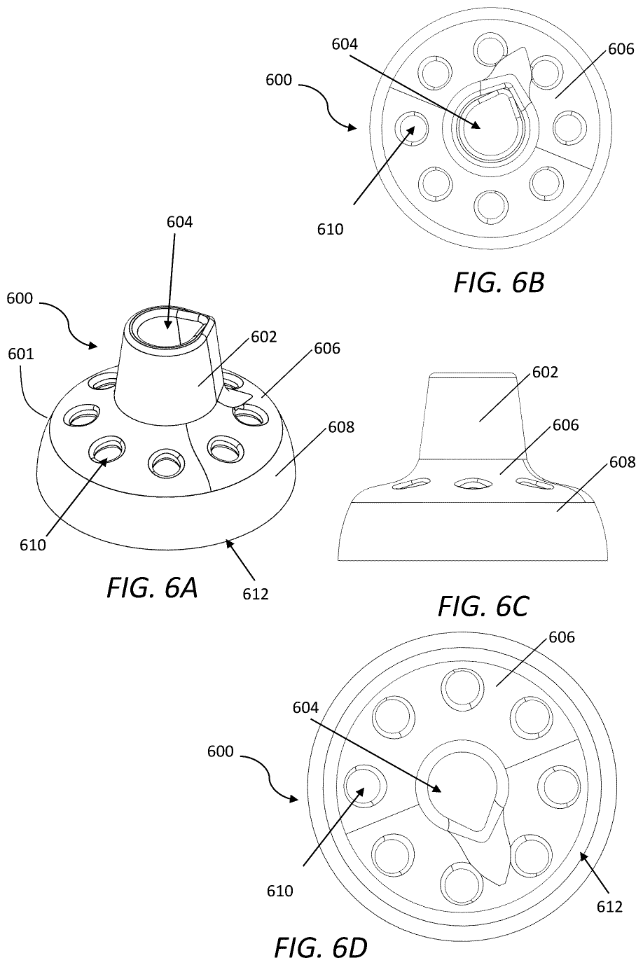

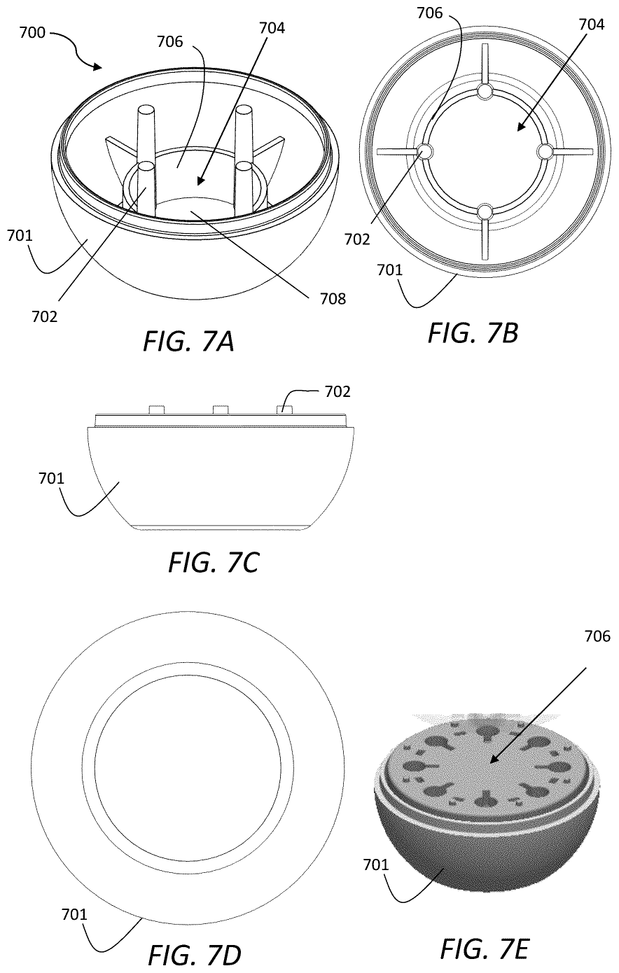

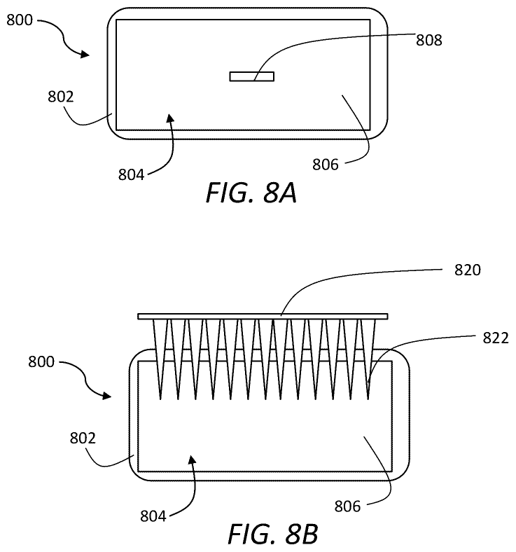

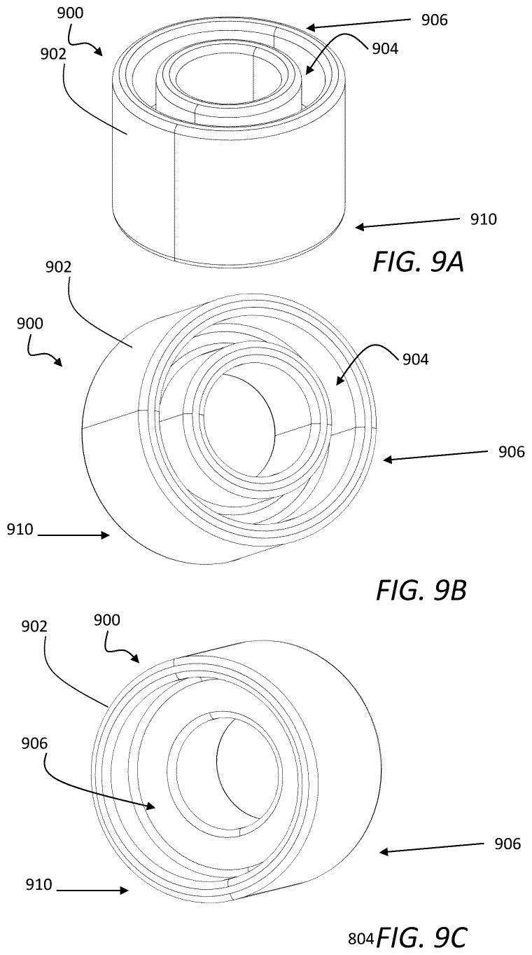

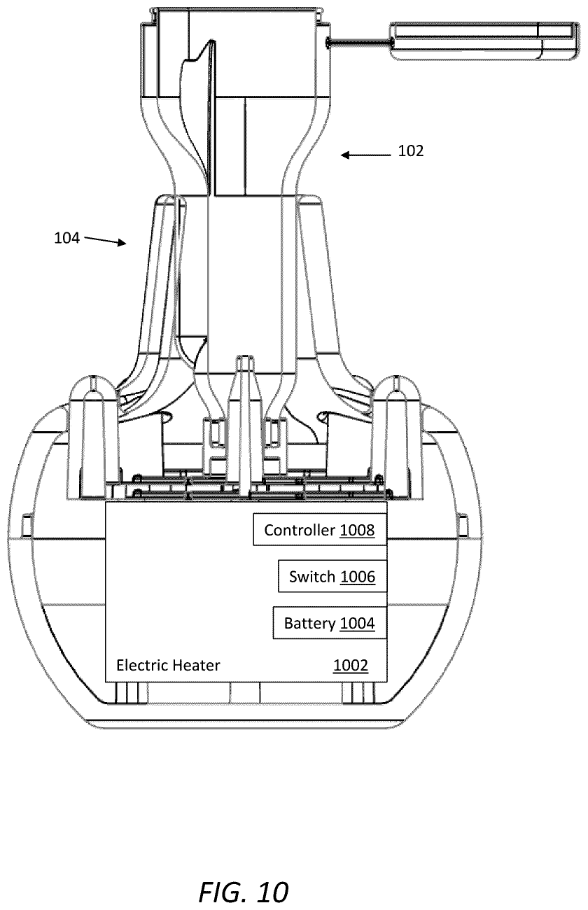

Also shown is the microfluidic reactor device 104 (i.e., microfluidic device 104) configured to be removably coupled with the sample tube 102. The external features of the microfluidic device 104 are shown in FIGS. 1A-1E with the sample tube 102 located therein. Cross-sectional view of the microfluidic device 104 retaining the sample tube 102 are shown in FIGS. 2A-2B. The microfluidic network member 300 of the microfluidic device 104 is shown in FIGS. 3A and 3C to be coupled with the sample tube 102. The sample tube 102 having an end cap 900 is shown in FIG. 3B. The microfluidic network member 300 is shown in FIGS. 5A-5D. FIGS. 6A-6D show the top cover 600 of the microfluidic device 104 and FIGS. 7A-7E show the bottom member of the microfluidic device 104. FIG. 8A illustrates a heating pouch 800 having the phase change material 806. FIG. 8B illustrates a pouch 802 that can be punctured with a material that induces a phase change in a phase change material 806. FIGS. 9A-9C illustrate the output cap 900, which can be an annular sample tube receiver when mounted in the microfluidic device 104. FIG. 10 shows an electric heater 1002 that can be powered by a battery 1004.

In some embodiments, the sample tube output end can be configured to be puncturable. As such, the output end includes a puncturable membrane or film material, such as an elastomer, rubber, or thermoplastic. The material may be a membrane or film over the output end. The sample tube 102 includes a port adapter 410 at the output end that is configured to couple with a port in the microfluidic device.

In some embodiments, the port adapter 410 can include a valve and the microfluidic device can include a mechanism so that rotation of the sample tube in one direction opens the valve and rotation in the other direction closes the valve. The end cap described herein can also include the valve, or it can include a mechanism for opening and closing the valve in the port adapter.

FIG. 3A shows that the microfluidic device 104 includes a port 310 for receiving the port adapter 410 of the sample tube 102. The port 310 includes a port member 502 and an annular receiver 312. As shown in FIG. 3B, the annular receiver 312 can function as an output cap that covers the output end 408. The port adapter 410 can fit into the annular receiver 312. FIG. 3A shows the sample distribution member 520 of the microfluidic device 104 includes the annular receiver 312 configured as an alignment port 312 having an annular recess configured for receiving an annular lip of the sample tube output end. That is, the annular receiver 312 can function as a cap 900 or as an alignment port 312. The alignment port 312 can be mounted in the microfluidic device 104, such as on the sample distribution member 520.

FIG. 3C shows the coupling of the port adapter 410 and the port member 502 without showing the annular receiver 312, which is present in the coupling. The annular receiver 312 can either be a cap as part of the sample tube 102 or as an alignment port 312 as part of the microfluidic device to guide coupling of the port adapter 410 onto the port member 502.

As shown, the sample tube 102 includes an open output end 408, and an output cap 900 is included to enclose the output end. The output cap 900 is removable from the output end 408, wherein the output cap 900 includes a puncturable membrane material that can be punctured with the tapered input tip of the port member. Also, the output cap 900 can have an annular recess (e.g., configured for receiving an annular lip 420 of the sample tube output end 408 as shown in FIG. 4D). FIGS. 9A-9C show an output cap 900 having the body 902 with the annular recess 904 in the sample tube end 906. The output cap 900 includes a recess 908 at the sample distribution member end 910.

FIGS. 5A-5D show the microfluidic device 104 that includes a port member 502 having a conical body with a tapered input tip 504. The tapered input tip 504 is configured to be coupled with the port adapter 410 of the output end 408 of the sample tube 102. The port member 502 includes an output end 506 with a port conduit 510 extending between a tip opening 512 at the input tip 504 to an output opening 514 at the output end 506. The tapered input tip 504 is configured to puncture the port adapter 410 of the sample tube 400 to obtain the sample from the port adapter 410 at the output end 408 of the sample tube 400. FIG. 5F shows another embodiment of the microfluidic device. In some embodiments, microfluidic device may be shaped in various shapes.

The microfluidic device 104 may include a sample distribution member 520 coupled with the output end 506 of the port member 502. In some embodiments, the sample distribution member 520 may include a microfluidic network 522 with one or more microfluidic channels 524 having a microfluidic channel inlet 526 fluidly coupled with the output opening 514 at the output end 506 of the port member 502. Each microfluidic channel 524 may further include a microfluidic channel outlet 528.

The microfluidic device 104 includes one or more reaction vessels 532 that are configured to contain reactions with the sample. One or more reaction chambers 530 may be located within the one or more reaction vessels 532 and be fluidly coupled with the microfluidic channel outlet 528 of the one or more microfluidic channels 524. Each reaction chamber 530 is in the reaction vessel 532 that also includes a readout region 534 that shows a colorimetric indicator. The reaction vessels 532 can include a lens 536 at the readout region and the reaction chambers 530 to facilitate viewing of the colorimetric indicator. In some embodiments, the reaction chamber 530 may be sized to contain certain amount of the sample and the reagents. For example, the reaction chamber 530 may be from about 10 μL to about 50 μL or from about 25 μL to about 40 μL.

FIG. 5E shows a fluidic seal 550 that is configured to be coupled with the sample distribution member 520 in order to fluidly seal the one or more microfluidic channels 524 of the microfluidic network 522. For example, the microfluidic channels 524 may be formed (e.g., molded, etched, etc.) in the top surface or bottom surface of the sample distribution member 520. The fluidic seal 550 may then be adhered with a pressure sensitive adhesive to that surface to cover the microfluidic channels 524 to be fluid tight between the microfluidic channel inlet 526 and microfluidic channel outlet 528, and thereby through the port conduit 510 to the tip opening 512.

FIG. 5F shows another embodiment of the sample distribution member 520 having the channels and reaction chambers 530. The fluidic seal 550 can be covered over the channels to make them fluid tight, and also cover the openings for the reaction chambers 530.

In some embodiments, the microfluidic device 104 also includes the top cover 600 as shown in FIGS. 6A-6D. As shown, the top cover 600 includes a neck 602 connected to a first upper annular portion 606 and a second annular portion 608. The neck 602 includes a main slot 604 configured to receive the sample tube 102 therein. The first upper annular portion 606 includes one or more openings 610 corresponding to the one or more reaction chambers 530. The one or more openings 610 may configured such that the one or more reaction chambers 532 are viewable through the one or more openings 610. In some embodiments, the one or more reaction chambers 532 and/or the reaction vessels 530 may extend at least partially through the one or more openings 610. The second annular portion 608 can include an engagement member 612 be configured to mate, such as threadedly coupled, with the bottom member 114 (FIG. 1A).

The microfluidic device 104 also includes a bottom member 700 with a bottom shell 701 configured to be mated with the top shell 601 of the top cover 600 to form the housing of the device. The housing can enclose the port member 502, the sample distribution member 520, and the fluidic seal 550. Also, the bottom member 700 includes support rods 702 to hold the sample distribution member 520. The bottom member 700 includes a bottom chamber 704 configured to retain a composition or electronic devices, such as an electronic heater and battery, therein. The bottom chamber 704 can include sidewalls 706 that extend up from a base 708. Optionally, the bottom member 700 can include a plate 710 that can hold the distribution member 520. The plate 710 can include aperture for the corresponding reaction vessels 532.

As shown in FIG. 5A, the port member 502 can have a conical body with a tapered input tip 504 and an output end 506 with a port conduit 510 extending between a tip opening 512 at the input tip 504 to an output opening 514 at the output end 506. The tapered input tip 504 is configured to connect with the sample tube 102 containing a sample by puncturing the sample tube at the adapter 410 or otherwise coupling therewith (e.g., via valve). For example, the port member 502 may obtain the sample contained in the sample tube 102 through the punctured portion of the sample tube 102 of via the valve. A sample distribution member 520 is coupled with the output end 514 of the port member 502. The sample distribution member 520 includes a microfluidic network 522 with one or more microfluidic channels 524 having a microfluidic channel inlet 526 fluidly coupled with the output opening 514 at the output end 506 of the port member 502. Each microfluidic channel 524 can include a microfluidic channel outlet 528. The sample distribution member 520 is configured to receive the sample from the port member 502 and distribute the sample through the one or more microfluidic channels 520 to the one or more reaction chambers 530. A fluidic seal 550 is coupled with the sample distribution member 520 to fluidly seal the one or more microfluidic channels 524 of the microfluidic network 522.

One or more reaction chambers 530 are fluidly coupled with the microfluidic channel outlets 528 of the one or more microfluidic channels 524. The one or more reaction chambers 530 are configured to receive the distributed sample from the microfluidic channel outlet 528 of the one or more microfluidic channels 524. Each reaction chamber 530 contains a reagent that is configured to interact with a target analyte in the sample. Each reaction vessel 532 includes a readout portion 534 to view the reagent interact with the target analyte. A top cover 600 is provided with a main slot 604 configured to receive the sample tube 102 therein. For example, the sample tube 102 may couple with the sample distribution member 520 through the main slot 604. In some embodiments, the top cover 600 may include one or more openings 610 that correspond to the one or more reaction chambers 532. The one or more openings 610 may allow observation of the readout regions of the one or more reaction chambers 532.

In some embodiments, a bottom member 700 of FIGS. 7A-7E may mate with the top cover 600 to enclose the port member 502, the sample distribution member 520, and the fluidic seal 550. The bottom member 700 may include support rods 702 holding up a platform member 706. The platform member 706 can be configured to hold the sample distribution member 520 above a bottom chamber 704 that contains a heating member, such as an electric heater or phase change material that can have an exothermic reaction, such as eutectic salt hydrate composition.

As described, a phase change material, such as a eutectic salt composition, can be configured to be placed into the bottom chamber 704. Such a phase change material is shown in FIGS. 8A-8B. Alternatively, the bottom chamber 704 can include an electric heater with batter (but could be plugged in) as shown in FIG. 10.

Additionally, a reagent can be configured to be included in each reaction chamber 530 that is adapted to provide a colorimetric reading upon interaction with a target analyte in the sample.

In some embodiments, the reagents in the reaction chamber 530 can be included in a matrix, powder, or other carrier material. For example, the reagents can be combined with a cellulose, such as carboxymethyl cellulose (CMC), or others. Alternatively, any material that can swell when contacted by liquid, where it allows airflow when dry and then swelling and preventing air flow when wet.

In some embodiments, each reaction vessel 532 may include a pin hole 550 in the end opposite of the inlet to the reaction chamber 530. This allows for reaction chambers 530 to release pressure, gasses, or fluids through the pin hole while the biological sample is filling into the reaction chamber 530 and interacting with the reagents and carrier. The carrier can hydrate and then plug the pin hole once the excess gas or fluid has been removed. For example, when the biological sample is flowing into the reaction chamber 530 the water portion (or other carrier liquid) can hydrate or fill the carrier and interact with the reagent. The gas in the reaction chamber (e.g., nitrogen, air, etc.) can be forced out the pin hole 550. This prevents pressure from building up in the reaction chamber 530, and also allows for easier flow, such as with wicking, or with active pumping (e.g., syringe fit into the sample tube receiver). For example, the pin hole can be microscopic or of significantly small dimension. The dimension is too small to allow any carrier or reagent spill from the reaction chamber 530.

In some embodiments, the system for analyzing a sample can include: a sample tube with an input end that is configured to receive the sample and having an attached lid that is adapted to seal the input end of the sample tube, and having an output end opposite of the input end, the output end having a port adapter; a microfluidic device configured to be removably coupled with the sample tube.

In some embodiments, the microfluidic device can include: a port member having a input tip adapted to couple with the port adapter of the output end of the sample tube, and having an output end with a port conduit extending between the input tip to the output end, wherein the input tip is configured to puncture the port adapter to obtain the sample from the port adapter at the output end of the sample tube; a sample distribution member coupled with the output end of the port member and having a microfluidic network with one or more microfluidic channels; one or more reaction chambers fluidly coupled with the one or more microfluidic channels, wherein each reaction chamber includes a readout region; a fluidic seal coupled with the sample distribution member to fluidly seal the one or more microfluidic channels of the microfluidic network; a top cover with first portion and a second portion, wherein the first portion includes a main slot configured to receive the sample tube therein, and the second portion includes one or more openings corresponding to the one or more reaction chambers; and a bottom member mating with the top cover to enclose the port member, sample distribution member, and the fluidic seal, and includes a bottom chamber configured to retain a composition. A heating member, such as an electric heater or phase change material can be located in the bottom chamber. A reagent can be located in at least one of the reaction chambers that is adapted to provide a colorimetric reading upon interaction with a target analyte in the sample. The plurality of reaction chambers allows for a plurality of different reactions. Also, each diagnostic reaction may include a positive and a control reading, which uses two reaction chambers for the diagnostic.

In some embodiments, a system for analyzing a sample can include: a sample tube with an input end that is configured to receive the sample and having an attached lid that is adapted to seal the input end of the sample tube, and having an output end opposite of the input end; a microfluidic device configured to be removably coupled with the sample tube. The microfluidic device can include: a port member, having an input end and an output end, configured to couple with the output end of the sample tube, wherein the input end is configured to obtain the sample from the output end of the sample tube; a sample distribution member coupled with the output end of the port member and having a microfluidic network with one or more microfluidic channels; one or more reaction chambers fluidly coupled with the one or more microfluidic channels, wherein each reaction chamber includes a readout region; a top cover with first portion and a second portion, wherein the first portion includes a main slot configured to receive the sample tube therein, and the second portion includes one or more openings corresponding to the one or more reaction chambers; and a bottom member mating with the top cover to enclose the port member and the sample distribution member, wherein the bottom member includes a bottom chamber configured to retain a heating member. The system can also include the heating member, such as electric heater or phase change material configured to be placed into the bottom chamber.

The phase change material can include a bladder having the contents of the composition, which when bent to expose a reagent or catalyst, the reaction occurs. The bladder can be configured and operate similar to a common hand warmer that is bent to activate the contents therein to generate the head. Also, a reagent can be located in each reaction chamber that is adapted to provide a colorimetric reading upon interaction with a target analyte in the sample.

In some embodiments, the system for analyzing a sample can include: a sample tube and having a removable lid and an output end; a microfluidic device comprising: a port member configured to couple with the output end of the sample tube; a sample distribution member coupled with the port member and having a microfluidic network with one or more microfluidic channels; one or more reaction chambers fluidly coupled with the one or more microfluidic channels, wherein each reaction chamber includes a readout region; a top cover with a main slot configured to receive the sample tube therein, and with one or more openings corresponding to the one or more reaction chambers; and a bottom member mating with the top cover to enclose the port member and the sample distribution member, wherein the bottom member includes a bottom chamber configured to retain a heating member.

In some embodiments, a method of manufacturing a sample analyzing system is provided. A bottom member can be obtained. The bottom member can include a bottom chamber having one or more support rods configured to support a bottom platform. A phase change material can be placed into the bottom chamber. A sample distribution member on the bottom platform can be placed on the bottom member. The sample distribution member can be coupled with a fluidic seal to fluidly seal one or more microfluidic channels of a microfluidic network within the sample distribution member. The sample distribution member also has a port member fluidly coupled with the one or more microfluidic channels. The port member can have a conical body with a tapered input tip.

One or more reaction chambers can be affixed to the sample distribution member, such as by being integrated therewith (e.g., molded, injection molded, and 3D printed, etc.) or as separate chambers coupled to the distribution member. The one or more reaction chambers are fluidly coupled with the one or more microfluidic channels. Each reaction chamber contains a reagent that is adapted to provide a colorimetric reading upon interaction with a target analyte. The one or more reaction chambers can be placed on top of the bottom platform.

The sample distribution member can be covered with a top cover. The top cover is configured to mate with the bottom member to enclose the port member, the sample distribution member, and the fluidic seal therein and form a housing. The top cover has a first upper portion and a second portion, wherein the first portion includes a main slot configured to receive a sample tube therein, and the second portion includes one or more openings corresponding to the one or more reaction chambers.

In some embodiments, the phase change material can be included in a container, such as a flexible bladder or bag. FIG. 8A illustrates an example of a heating pouch 800 having a flexible polymeric body 802 defining a cavity 804 having a phase change material 806 therein. An activator 808 is included in the cavity 804 with the phase change material 806. The activator can include a material that activates the phase change material to change from liquid to solid, which is an exothermic reaction that produced heat. The activator can be a capsule or container with the activating material therein. The activating material can be chosen depending on the type of phase change material, such as the hydrate composition. For example, the activating material can be a crystal of the salt hydrate that causes a phase change from liquid to solid. Another example can be oxygen, iron, or the like. The activator 808 can be activated by bending the body 802 to bend or break the activator 808 so as that the activating material interacts with the phase change material 806.

FIG. 8B shows the heating pouch 800, which is devoid of the activator 808. Instead, the device includes the fluidic seal 550 or member 820 attached thereto having at least one protrusion 822, such as a spike, that penetrates into the cavity 804 to contact the phase change material 806. This exposure to oxygen or contact with the protrusion 822 can induce the crystallization of a eutectic salt hydrate as the phase change material 806. This penetration into the phase change material 806 can include the crystallization or phase change that is exothermic and generates heat. In some embodiments, a crystal of the phase change material can be coated onto the protrusion 822 so as to contact the phase change material 806 after penetration. In some embodiments, by pushing the sample tube 102 into the cartridge 104 can cause the protrusion 822 to penetrate the cavity 804 and contact the phase change material 806 to induce the phase change and heating. In some aspects, the protrusion 822 can be made from or coated with a nucleating material that nucleates the phase change material to cause the phase change and crystallization.

In some embodiments, the phase change material is as salt hydrate, which can be a eutectic salt hydrate to provide the heating. A eutectic salt hydrate is a mixture of hydrated salts in water (or potentially other solvent). These salts may be “super cooled” meaning they remain a liquid below their freezing point, where they can be included in the heating pouch. When ‘activated’, such as via introduction of a salt crystal from the activator, a phase change occurs turning the hydrate form liquid into solid. In undergoing the phase change from liquid to solid, the salt hydrate heats to its phase transition temperature. For example, sodium acetate trihydrate (most common) can heat to precisely 58° C. A eutectic mixture enables the precise tuning of the phase transition point so that a desired temperature can be set. There may be specific temperatures or temperature ranges that are useful for diagnostic reactions, and thereby the eutectic salt hydrate can be tuned to obtain that suitable temperature. Table 1 provides examples of eutectic salt hydrates and the phase transition temperature (e.g., melting temperatures) thereof.

TABLE 1
Eutectic Salt Hydrates

| | Phase Transition | |
|---|---|---|
| Salt Hydrate | Temperature | |
| Sodium Acetate Trihydrate | 58° | C. |
| 50% Mg(NO₃)₂ 6H₂O + 50% Al(NO₃)₂ | 65° | C. |
| 9H₂O | | |
| Mg(NO₃)₂ 6H₂O | 89.9° | C. |
| Al(NO₃)₂ 8H₂O | 89.3° | C. |
| MgCl₂ 6H₂O | 117° | C. |

The temperature of the hydrate salt system can be tailored to obtain certain temperature ranges needed for a diagnostic reaction. For example, the 50% Mg(NO₃)₂ 6H₂O+50% Al(NO₃)₂ 9H₂O achieves 65° C. However, variations in the ratio can alter the temperature from 60-65° C.

For example, a target heating range between 30° C. and 65° C. can be obtained by modulating the salt hydrates and the compositions thereof.

In some embodiments, the device can include an electric heater. FIG. 10 shows the microfluidic device 104 having an electric heater 1002. Accordingly, the electric heater 1102 may include a battery 1004 or it can include a port to receive a power cord, or include an integrated power cord. Also, a switch 1006 can be included to turn the electric heater on or off. Also, a controller 1008 (e.g., computer, edge computer, control circuitry, etc.) can be included to control the temperature and/or time of maintaining temperature and any temperature ramping.

An example of reactions can be for detection of sexually transmitted diseases (STD). For example, the reaction chambers can include reagents that react with gonorrhea, Chlamydia, trichomonas, human papilloma virus (HPV) to provide for the diagnostics.

In another example, CRISPR reagents can be included within the reaction chambers to perform CRISPR analysis.

Another example includes assay reagents for hypophosphatasia (HPP).

Another example includes assay reagents to detect a virus, such as flu, covid, or others.

The reaction chambers of the microfluidic device can include reagents to react with a target analyte when present. This allows for colorimetric assays, which provide a defined color when the target analyte is present.

The biological sample can be any type from a swab in the nose, mouth, penis tip, vagina, anus, or other location. The swab can be placed into the sample tube, and a sample buffer can be introduced therewith. The sample buffer can transport the target analytes from the swab to the reaction chambers for the diagnostics.

In some examples, the biological sample can be a fluid such as blood, plasma, or extract therefrom. The blood can be processed to obtain fractions thereof or components thereof. The plasma may also be separated from the blood and used in the sample tube.

In some examples, the biological sample can be urine.

In some examples, the biological sample can be hair, where a solvent is added as a sample solvent to carry the target analyte.

In some examples, the biological sample can be feces, where the sample buffer can be used to obtain target analyte therefrom. The sample may be filtered before being introduced into the sample tube.

The sample tube and cartridge can be provided together as a kit. The cartridge can be the microfluidic reaction device descried herein that includes reaction reagents in the reaction chambers thereof. The microfluidics move the sample from the tube to the reaction chambers for diagnostic reactions. The kit can also include the heating pouch with the eutectic salt hydrate, where the eutectic salt hydrate is configured to heat to a temperature range sufficient for the reaction reagents to react with the target analyte in a sample.

In some embodiments, the sample is from a swab.

In some embodiments, a method of analyzing a sample, such as from a swab, is provided. The method can include obtaining a sample in a sample tube. The sample is put into the tube at an input end with an input opening. The sample tube also has an output end having a port adapter. The port adapter configured to release the sample in response to being punctured or otherwise coupling with the assay cartridge. The port adapter of the sample tube can be connected to the port member of a microfluidic device of the cartridge. The port member can have a conical body with a tapered input tip adapted to couple with the port adapter, and having an output end with a port conduit extending between a tip opening at the input tip to an output opening at the output end. The tapered input tip can be configured to puncture the port adapter to obtain the sample from the sample tube. Alternatively, the tapered input tip can be configured to fluidly couple with the port adapter to receive the contents of the sample tube therefrom. The sample can be distributed to one or more reaction chambers via the microfluidic network of the cartridge. The sample may use wicking to draw from the reaction tube to the reaction chambers. Also, a sample buffer can be introduced into the sample tube to carry the sample and target analyte therein through the port into the microfluidic network and to the reaction chambers. The sample distribution member can have a microfluidic network with one or more microfluidic channels. The one or more microfluidic channels can have a microfluidic channel inlet fluidly coupled with the output opening at the output end of the port member. Also, the channels can each include a microfluidic channel outlet fluidly coupled with the one or more reaction chambers.

Heat can be provided to the one or more reaction chambers by an electric heater or eutectic salt composition that is located in the chamber of the cartridge, which is below the reaction chambers. The heating member can be placed in a bottom chamber under the one or more reaction chambers. The eutectic salt hydrate can be in a package, such as in a heating pouch that is a plastic bladder containing the liquid eutectic salt hydrate. For example, the heating pouch can be activated and then placed in the chamber. In another example, the heating pouch may already be in the chamber of the cartridge, and the pressing of the sample tube into the cartridge can interact with the heating pouch for activation. The electric heater may include a switch that can be actuated to turn on the heat.

The distributed sample then interacts with a reagent pre-deposited in each reaction chamber under heat from the heating pouch. The reagent can be adapted to provide a colorimetric reading upon interaction with a target analyte in the sample. This allows for the reading of the colorimetric color that is generated from a reaction of the target analyte with the reaction reagent in each reaction chamber. The colorimetric color can be observed through a readout region of each reaction chamber.

The reagent can be in a lyophilized powder with the carrier, and configured to be retained in the reaction chamber without passing through the pin hole. A pressure sensitive adhesive can be used for a backing to cover the channels and thereby provide a cover for the reaction chambers. This can keep the reagent in the reaction chamber without falling out.

In some embodiments, the cartridge can include a lever that is pushed to actuate an arm to activate the heating pouch. The arm can press the activator in the pouch so that the activator interacts with the hydrate salt for inducing the phase transition. In another example, pressing the sample tube into the cartridge can press an arm into the heating pouch to activate the activator and induce the phase changes. Other ways of activating the phase change to generate heat from the salt hydrate are also contemplated.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.

1. A system for analyzing a sample comprising:
a sample tube having an input end removably covered using a removable lid and an output end;
a microfluidic device comprising:
a port member configured to couple with the output end of the sample tube;
a sample distribution member coupled with the port member and having a microfluidic network with one or more microfluidic channels;
one or more reaction chambers fluidly coupled with the one or more microfluidic channels, wherein the reaction chambers include readout regions corresponding to each reaction chamber;
a top cover with a main slot configured to receive the sample tube therein, and with one or more openings corresponding to the one or more reaction chambers; and
a bottom member mating with the top cover to enclose the port member and the sample distribution member, wherein the bottom member includes a bottom chamber configured to retain a composition;
a heating member configured to be placed into the bottom chamber; and
a reagent in reaction chamber that is adapted to provide a colorimetric reading upon interaction with a target analyte in the sample.

2. The system of claim 1, wherein the port member has a conical body with a tapered input tip.

3. The system of claim 2, wherein the output end of the sample tube includes a port adapter configured to couple with a port of the microfluidic device.

4. The system of claim 3, wherein the output end of the sample tube includes a film covering the port adapter that includes a puncturable membrane material that can be punctured with the tapered input tip of the port member, such as an elastomer, rubber, or thermoplastic.

5. The system of claim 3, wherein the port includes the port member and an annular receiver.

6. The system of claim 5, wherein the annular receiver is configured as an alignment port having an annular recess configured for receiving an annular lip of the output end of the sample tube.

7. The system of claim 1, wherein the sample tube is configured for receiving a swab, the sample tube being configured for passing content of the swab to the port member.

8. The system of claim 1, wherein the sample tube includes a spout at the input end, wherein the lid is configured to cover the spout.

9. The system of claim 1, wherein the microfluidic device includes an even number of the reaction chambers.

10. The system of claim 1, wherein the reaction chambers protrude from the sample distribution member through one or more openings such that the readout regions are viewable through the one or more openings.

11. The system of claim 1, further comprising a plunger adapted to fit into the sample tube and function as a syringe for delivering the sample through the microfluidic network to the reaction chambers.

12. The system of claim 1, wherein the reagent is configured to test for a sexually transmitted disease with a colorimetric assay.

13. The system of claim 1, wherein the heating member is a eutectic salt composition including a salt hydrate having a phase transition temperature between about 37° C. and about 90° C.

14. The system of claim 13, wherein the salt hydrate is: 50% Mg(NO₃)₂ 6H₂O+50% Al(NO₃)₂ 9H₂O with a phase transition temperature of about 65° C.

15. The system of claim 1, wherein the heating member is an electronic heater.

16. A method of analyzing a sample comprising:
obtaining a sample in a sample tube, the sample tube having an input end with an input opening where the sample is obtained through, the sample tube also having an output end having a port adapter, the port adapter configured to release the sample in response to being punctured;
attaching the port adapter of the sample tube to a port member of a microfluidic device, the port member having a conical body with a tapered input tip adapted to couple with the port adapter, and having an output end with a port conduit extending between a tip opening at the input tip to an output opening at the output end, wherein the tapered input tip is configured to puncture the port adapter to obtain the sample from the sample tube;
distributing the sample to one or more reaction chambers, by a sample distribution member having a microfluidic network with one or more microfluidic channels, the one or more microfluidic channels having a microfluidic channel inlet fluidly coupled with the output opening at the output end of the port member, and having a microfluidic channel outlet fluidly coupled with the one or more reaction chambers;
providing heat to the one or more reaction chambers by a heating member in a bottom chamber under the one or more reaction chambers;
interacting the distributed sample with a reagent pre-deposited in each reaction chamber, the reagent adapted to provide a colorimetric reading upon interaction with a target analyte in the sample; and
reading the colorimetric reading of each reaction chamber through a readout region of each reaction chamber.

17. The method of claim 16, wherein the sample is obtained using a swab, the swab used to obtain the sample from a subject.

18. The method of claim 16, wherein the heat is provided to the one or more reaction chambers using a heating member.

19. The method of claim 18, wherein the heating member is:
a eutectic salt composition including a salt hydrate having a phase transition temperature between about 37° C. and about 90° C.; or
an electronic heater.

20. A method of manufacturing a sample analyzing system comprising:
obtaining a bottom member, the bottom member including a bottom chamber, the bottom chamber surrounded by one or more support rods configured to support a bottom platform;
placing a heating member into the bottom chamber;
placing a sample distribution member on the bottom platform, the sample distribution member coupled with a fluidic seal to fluidly seal one or more microfluidic channels of a microfluidic network within the sample distribution member, the sample distribution member also having a port member fluidly coupled with the one or more microfluidic channels, the port member having a conical body with a tapered input tip;
attaching one or more reaction chambers to the sample distribution member, the one or more reaction chambers fluidly coupled with the one or more microfluidic channels, each reaction chamber containing a reagent that is adapted to provide a colorimetric reading upon interaction with a target analyte, the one or more reaction chambers also placed on top of the bottom platform;
covering the sample distribution member with a top cover, the top cover configured to mate with the bottom member to enclose the port member, the sample distribution member, and the fluidic seal, the top cover having a first portion and a second portion, wherein the first portion includes a main slot configured to receive a sample tube therein, and the second portion includes one or more openings corresponding to the one or more reaction chambers; and
placing the sample tube through the main slot of the top cover to be removably coupled with the port member, the sample tube having an input end and an output end, the sample tube configured to obtain a sample to be analyzed through the input end, wherein the port member may connect with the output end of the sample tube, the port member configured to distribute the sample to the one or more reaction chambers through the sample distribution member.
